(19) United States
(12) Patent Application Publication
(10) Pub. No.: US 2016/0023008 A1
(43) Pub. Date: Jan. 28, 2016

(54) SYSTEMS AND METHODS FOR SYNCHRONIZING STIMULATION DATA

(71) Applicant: Boston Scientific Neuromodulation Corporation, Valencia, CA (US)

(72) Inventor: Sridhar Kothandaraman, Valencia, CA (US)

(21) Appl. No.: 14/807,499

(22) Filed: Jul. 23, 2015

Related U.S. Application Data
(60) Provisional application No. 62/028,688, filed on Jul. 24, 2014.

(51) Int. Cl. A61N 1/372

(52) U.S. Cl. CPC A61N 1/37247; A61N 1/37264; A61N 1/37217

(57) ABSTRACT

An electrical stimulation system includes an implantable control module for implantation in a body of a patient. The control module includes an antenna and a processor coupled to the antenna. The control module can provide electrical stimulation signals to an electrical stimulation lead coupled to the implantable control module for stimulation of patient tissue. The system also includes a first external programming unit to communicate with the processor of the implantable control module using the antenna and to provide or update stimulation parameters for production of the electrical stimulation signals. The first external programming unit also communicates with a data storage unit remote from the first external programming unit and the implantable control module to store the stimulation parameters at the data storage unit and retrieve the stimulation parameters from the data storage unit. Optionally, the system also includes the data storage unit.

### CROSS-REFERENCE TO RELATED APPLICATIONS

This application claims the benefit under 35 U.S.C. §119(e) of U.S. Provisional Patent Application Ser. No. 62/028,688 filed Jul. 24, 2014, which is incorporated herein by reference.

### FIELD

The present invention is directed to the area of implantable electrical stimulation systems and methods of making and using the systems. The present invention is also directed to implantable electrical stimulation systems that use remote data storage for stimulation parameters that can be accessed by multiple programming units, as well as methods of making and using the electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include an implantable pulse generator (IPG), one or more leads, and an array of stimulator electrodes on each lead. The stimulator electrodes are in contact with or near the nerves, muscles, or other tissue to be stimulated. The pulse generator generates electrical pulses that are delivered by the electrodes to body tissue.

### BRIEF SUMMARY

One embodiment is an electrical stimulation system that includes an implantable control module configured and arranged for implantation in a body of a patient. The control module includes an antenna and a processor coupled to the antenna. The control module is configured and arranged to provide electrical stimulation signals to an electrical stimulation lead coupled to the implantable control module for stimulation of patient tissue. The system also includes a first external programming unit configured and arranged to communicate with the processor of the implantable control module using the antenna and to provide or update stimulation parameters for production of the electrical stimulation signals. The first external programming unit is configured and arranged to communicate with a data storage unit remote from the first external programming unit and the implantable control module to store the stimulation parameters at the data storage unit and retrieve the stimulation parameters from the data storage unit.

Optionally, the system also includes the data storage unit. In at least some embodiments, the control module and data storage unit are configured and arranged to communicate directly with each other. In at least some embodiments, the system also includes a second external programming unit configured and arranged to communicate with the control module and the data storage unit and to provide or update stimulation parameters for production of the electrical stimulation signals. In at least some embodiments, the data storage unit is a second external programming unit that is configured and arranged to communicate with the processor of the implantable control module using the antenna and to provide or update stimulation parameters for production of the electrical stimulation signals.

In at least some embodiments, the external programming unit includes a user interface configured and arranged to receive input from a user, and a processor in communication with the user interface and configured and arranged to perform the following actions: a) receiving input from the user to change or add at least one stimulation parameter, b) in response to receiving the input, communicating the at least one stimulation parameter to the implantable control module, and c) in response to receiving the input, communicating the at least one stimulation parameter to the data storage unit for storing or updating the at least one stimulation parameter. In at least some embodiments, the processor is configured and arranged to perform the following additional action: d) retrieving a plurality of stimulation parameters from the data storage unit in preparation for receiving input from the user regarding at least one stimulation parameter.

In at least some embodiments, the data storage unit includes a memory, and a processor in communication with the memory and configured and arranged to perform the following actions: a) requesting stimulation data from at least one external programming unit, wherein the stimulation data comprises at least one stimulation parameter, b) in response to requesting the stimulation data, receiving the stimulation data from the at least one external programming unit, and c) in response to receiving the stimulation data, using the stimulation data to update stimulation data stored in the memory. In at least some embodiments, the action of requesting stimulation data includes requesting the stimulation data from at least one external programming unit on a regular periodic basis. In at least some embodiments, the action of requesting stimulation data includes requesting the stimulation data from at least one external programming unit in response to a triggering event. In at least some embodiments, the processor is configured and arranged to perform the following additional action: d) in response to receiving the stimulation data, communicating at least a portion of the stimulation data to at least one of the at least one external programming unit.

Another embodiment is an external programming unit that includes a user interface configured and arranged to receive input from a user, and a processor in communication with the user interface and configured and arranged to perform the following actions: a) receiving input from the user to change or add at least one stimulation parameter, b) in response to receiving the input, communicating the at least one stimulation parameter to the implantable control module, and c) in response to receiving the input, communicating the at least one stimulation parameter to the data storage unit for storing or updating the at least one stimulation parameter.

A further embodiment is a data storage unit that includes a memory, and a processor in communication with the memory and configured and arranged to perform the following actions: a) requesting stimulation data from at least one external programming unit, wherein the stimulation data comprises at least one stimulation parameter, b) in response to requesting the stimulation data, receiving the stimulation data from the at least one external programming unit, and c) in response to receiving the stimulation data, using the stimulation data to update stimulation data stored in the memory.

Yet another embodiment is a non-transitory computer-readable medium having processor-executable instructions for providing or updating stimulation parameters of an electrical stimulation system, the processor-executable instructions when installed onto a device enable the device to perform actions, including: receiving input from the user to change or add at least one stimulation parameter; in response to receiving the input, communicating the at least one stimulation parameter to an implantable control module; and, in response to receiving the input, communicating the at least one stimulation parameter to a data storage unit for storing or updating the at least one stimulation parameter. The data storage unit is remote from the control module and from the device performing the actions.

In at least some embodiments, the processor-executable instructions when installed onto a device enable the device to perform the following additional action: retrieving a plurality of stimulation parameters from the data storage unit in preparation for receiving input from the user regarding at least one stimulation parameter. In at least some embodiments, retrieving a plurality of stimulation parameters includes requesting an update of the plurality of stimulation parameters with a date of a last update and retrieving those stimulation parameters that have changed since the date of the last update.

Another embodiment is a non-transitory computer-readable medium having processor-executable instructions for providing or updating stimulation parameters of an electrical stimulation system, the processor-executable instructions when installed onto a device enable the device to perform actions, including: requesting stimulation data from at least one external programming unit, wherein the stimulation data comprises at least one stimulation parameter; in response to requesting the stimulation data, receiving the stimulation data from the at least one external programming unit; and, in response to receiving the stimulation data, using the stimulation data to update stimulation data stored in the memory.

In at least some embodiments, the action of requesting stimulation data includes requesting the stimulation data from at least one external programming unit on a regular periodic basis. In at least some embodiments, the action of requesting stimulation data comprises requesting the stimulation data from at least one external programming unit in response to a triggering event. In at least some embodiments, the processor-executable instructions when installed onto a device enable the device to perform the following additional action: in response to receiving the stimulation data, communicating at least a portion of the stimulation data to at least one of the at least one external programming unit. In at least some embodiments, the processor-executable instructions when installed onto a device enable the device to perform the following additional action: maintaining a history of changes to the stimulation data. In at least some embodiments, the processor-executable instructions when installed onto a device enable the device to perform the following additional action: in response to requesting the stimulation data, if an external programming unit does not respond, sending a follow-up request for the stimulation data.

A further embodiment is a method for providing or updating stimulation parameters that includes receiving input from the user to change or add at least one stimulation parameter; in response to receiving the input, communicating the at least one stimulation parameter to an implantable control module; and, in response to receiving the input, communicating the at least one stimulation parameter to a data storage unit for storing or updating the at least one stimulation parameter. The remote data storage unit is remote from the control module and from the device performing the actions.

Yet another embodiment is a method for providing or updating stimulation parameters of an electrical stimulation system that includes requesting stimulation data from at least one external programming unit, wherein the stimulation data comprises at least one stimulation parameter; in response to requesting the stimulation data, receiving the stimulation data from the at least one external programming unit; and, in response to receiving the stimulation data, using the stimulation data to update stimulation data stored in the memory.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic block diagram of one embodiment of an electrical stimulation system, according to the invention;

FIG. 2 is a schematic block diagram of another embodiment of an electrical stimulation system, according to the invention;

FIG. 3 is a schematic block diagram of another embodiment of an electrical stimulation system, according to the invention;

FIG. 4 is a schematic block diagram of one embodiment of a remote data storage unit, according to the invention;

FIG. 5 is a schematic block diagram of one embodiment of an external programming unit, according to the invention:

FIG. 6 is a flowchart of one embodiment of a method for updating or synchronizing stimulation data from an external programming unit to a remote data storage unit, according to the invention;

FIG. 7 is a flowchart of one embodiment of a method for updating or synchronizing stimulation data from a remote data storage unit to an external programming unit, according to the invention:

FIG. 8 is a flowchart of one embodiment of a method for updating or synchronizing stimulation data on a remote data storage unit from one or more external programming units, according to the invention;

FIG. 9 is a flowchart of another embodiment of a method for updating or synchronizing stimulation data on a remote data storage unit from one or more external programming units, according to the invention;

FIG. 10 is a schematic side view of one embodiment of a lead and control module, according to the invention;

FIG. 11 is a schematic side view of another embodiment of a lead and control module, according to the invention;

FIG. 12A is a schematic side view of one embodiment of an implantable control module configured and arranged to electrically couple to an elongated device, according to the invention; and

FIG. 12B is a schematic side view of one embodiment of a lead extension configured and arranged to electrically couple an elongated device to an implantable control module, according to the invention.

### DETAILED DESCRIPTION

The present invention is directed to the area of implantable electrical stimulation systems and methods of making and using the systems. The present invention is also directed to implantable electrical stimulation systems that use remote data storage for stimulation parameters that can be accessed by multiple programming units, as well as methods of making and using the electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364.278; and U.S. Patent Application Publication No. 2007/0150036, all of which are incorporated by reference.

In many conventional electrical stimulation systems, the lead is coupled to an implantable control module (e.g., an implantable pulse generator) that can be programmed by a clinician using an external programming unit. A clinician's office may have multiple external programming units. Conventionally, where multiple external programming units may be in use, each time a clinician connects to the control module, the external programming unit downloads all of the stimulation parameters and optionally other data from the control module. The time it takes to download this data may be significant as the control module may have limited communications abilities, speed, or bandwidth.

In contrast to such electrical stimulation systems, an electrical stimulation system can include a remote data storage unit, other than the control module, that can be accessed by an external programming unit to obtain the current stimulation parameters and optionally other data for programming the control module and which also receives and stores the stimulation parameters and any changes to the stimulation parameters and other data for later access. This system can also be useful for programming a replacement control module if the original control module fails or is otherwise replaced. The system can be any electrical stimulation system including any implantable system for spinal cord stimulation, deep brain stimulation, or stimulation of other tissue (including, but not limited to, neural tissue) or organ.

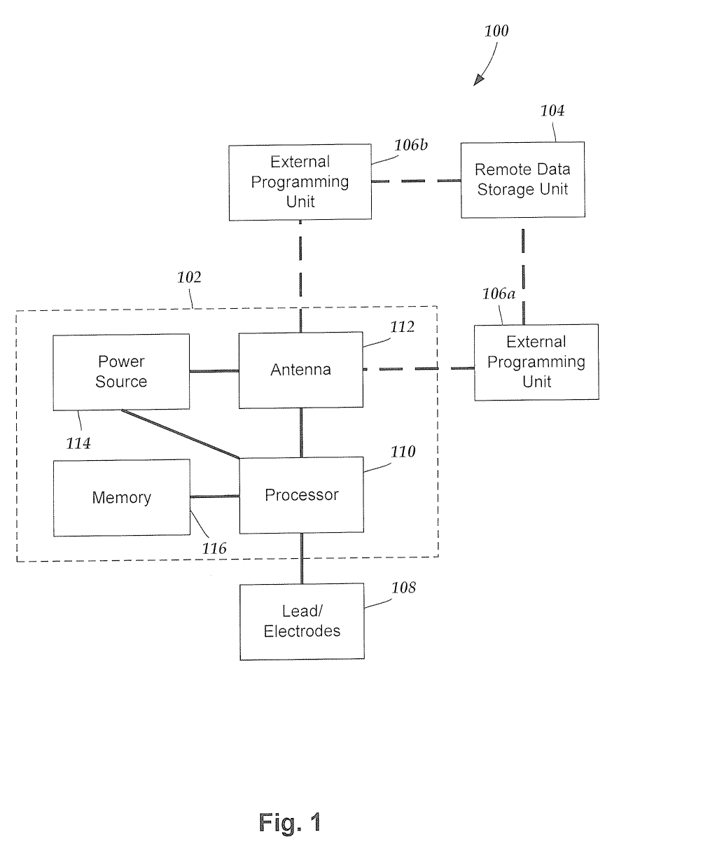

FIG. 1 illustrates schematically one embodiment of an electrical stimulation system 100 that includes an implantable control module (e.g., an implantable electrical stimulator or implantable pulse generator) 102, one or more leads 108 with electrodes, one or more external programming unit 106a, 106b, and a remote data storage unit 104. Alternatively, the implantable control module 102 can be part of a microstimulator with the electrodes disposed on the housing of the microstimulator. The microstimulator may not include a lead or, in other embodiments, a lead may extend from the microstimulator. It will be understood that the electrical stimulation system can include more, fewer, or different components and can have a variety of different configurations including those configurations disclosed in the references cited herein. For example, although FIG. 1 illustrates two external programming units 106a, 106b, one control module 102, and one remote data storage unit 104, it will be understood that the system can include more than two external programming units, more than one control module, and more than one remote data storage unit. In at least some embodiments, a clinician, user, or other individual may associate a particular control module 102 with one or more external programming units 106a, 106b and one or more remote data storage units 104. This association may be updated or otherwise changed. In other embodiments, the association of a control module with an external programming unit or remote data storage unit may be automatic.

The lead 108 is coupled, or coupleable, to the implantable control module 102. The implantable control module 102 includes a processor 110, an antenna 112 (or other communications arrangement), a power source 114, and a memory 116, as illustrated in FIG. 1.

One example of an external programming unit 106 is illustrated in FIG. 5 and includes a processor 150, a memory 152, a communications arrangement 154 (such as an antenna or any other suitable communications device such as those described below), and a user interface 156. Suitable devices for use as an external programming unit can include, but are not limited to, a computer, a tablet, a mobile telephone, a personal desk assistant, a dedicated device for external programming, remote control, or the like.

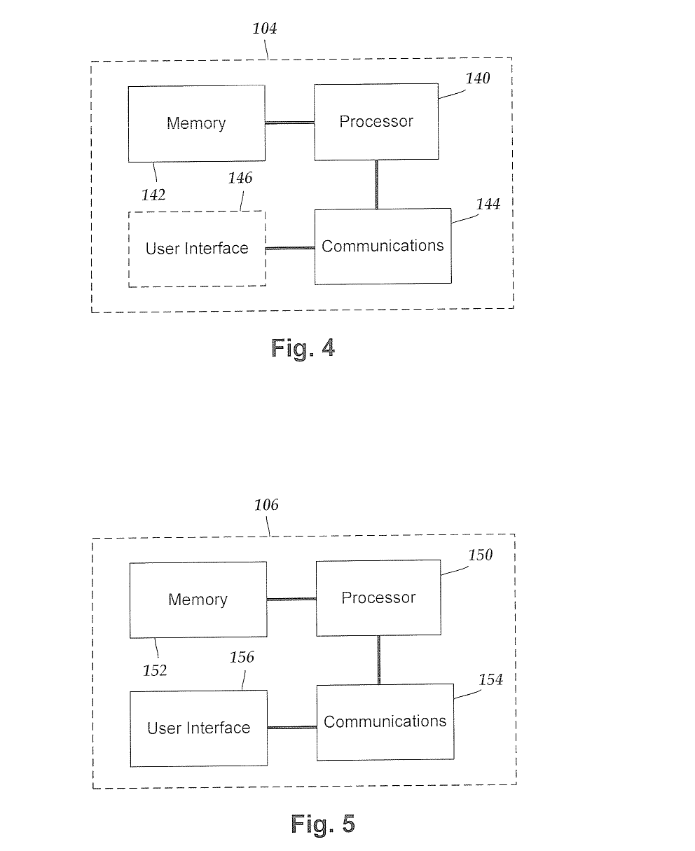

One example of a remote data storage unit 104 is illustrated in FIG. 4 and includes a processor 140, a memory 142, a communications arrangement 144 (such as an antenna or any other suitable communications device such as those described below), and an optional user interface 146. Suitable devices for use as a remote data storage unit can include, but are not limited to, a computer, a tablet, a server or server farm, a dedicated device for data storage, a hard drive, cloud storage arrangement, or the like. It will be understood that the external programming unit 106 and remote data storage unit 104 can include a power supply or receive power from an external source or any combination thereof. In at least some embodiments, the remote data storage unit 104 is physically separate from the control module 102 and the external program unit 106 and may be in communication with one or more of these devices through a local or wide area network or any other suitable communication arrangement.

In some embodiments, the remote data storage unit 104 can also act as a database to, for example, assist in providing electrical stimulation therapy feedback. Examples of databases and their use and operation in electrical stimulation systems, as well as the use of a separate patient interface unit (which can be added to any of the embodiments described herein) can be found in U.S. Provisional Patent Application Ser. No. ______, entitled “Systems, Devices, and Methods for Providing Electrical Stimulation Therapy Feedback”, filed Jul. 24, 2014 (Attorney Docket No. BSNC-1-374.0), incorporated herein by reference.

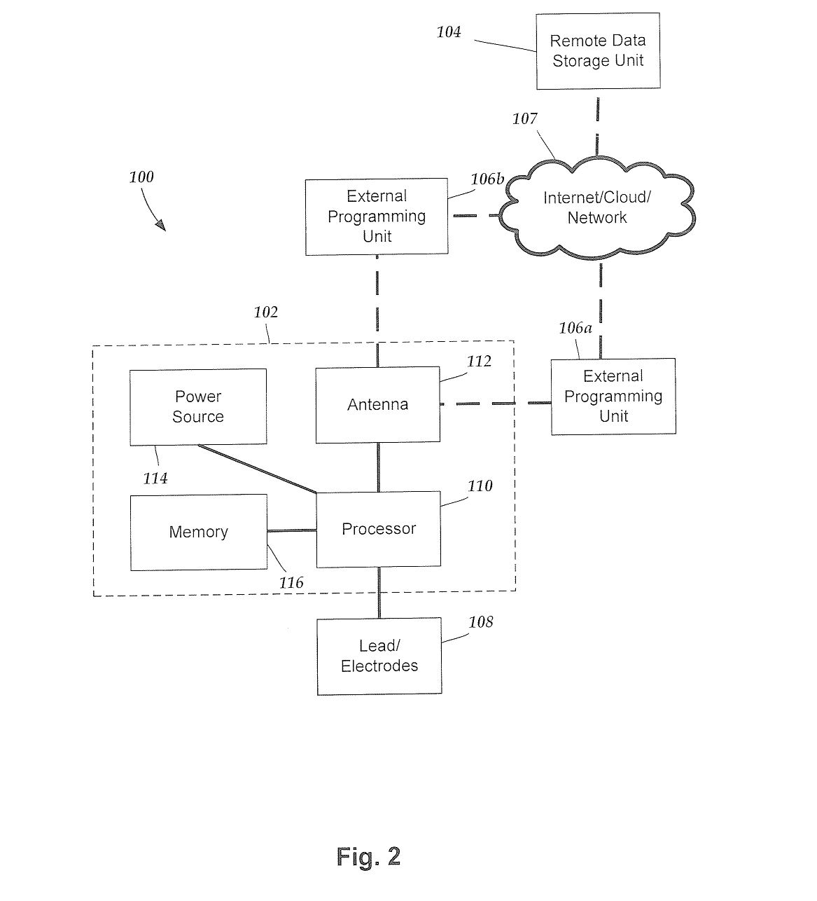

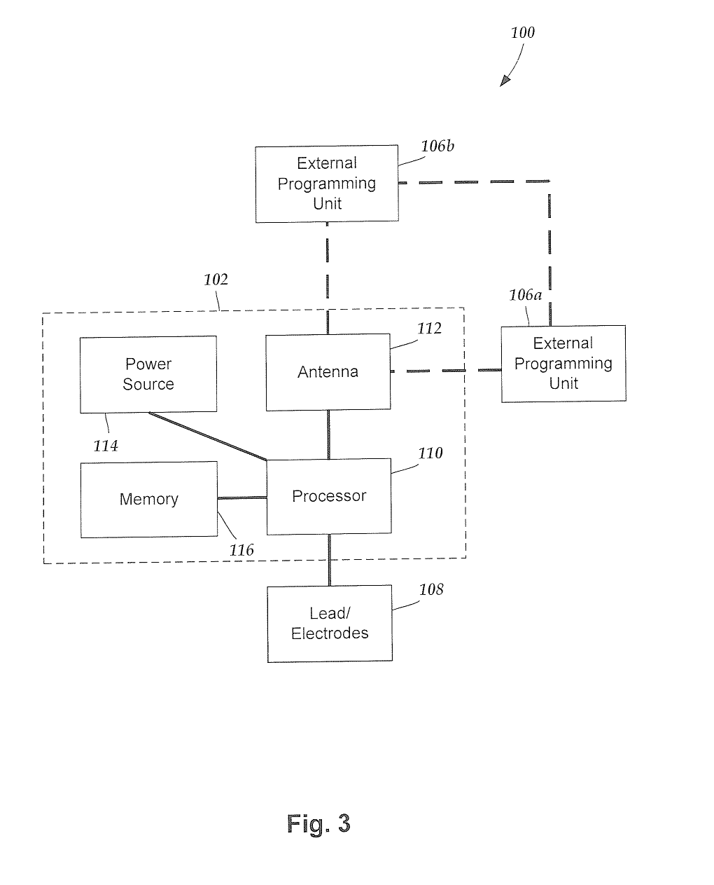

In the embodiment of FIG. 1, the external programming units 106a, 106b communicate directly with the remote data storage unit 104. FIG. 2 illustrates another embodiment in which the external programming units 106a, 106b communicate through the Internet, a cloud, or a local or wide area network 107 (including wireless local or wide area networks) or any combination thereof, or any other suitable intermediary or combination of intermediaries, to the remote data storage unit 104. FIG. 3 illustrates yet another embodiment in which one or more of the external programming units 106a also acts as the data storage unit and the other external programming units (for example, external programming unit 106b) communicate with it. In some embodiments (such as those in FIGS. 1 and 2), the remote data storage unit 104 can also communicate directly, or through an intermediary, such as the Internet, a cloud, or a local or wide area network 107, with the control module 102.

Methods of communication between devices or components of a system can include wired or wireless (e.g., RF, optical, infrared, near field communication (NFC), Bluetooth™, or the like) communications methods or any combination thereof. By way of further example, communication methods can be performed using any type of communication media or any combination of communication media including, but not limited to, wired media such as twisted pair, coaxial cable, fiber optics, wave guides, and other wired media and wireless media such as acoustic, RF, optical, infrared, NFC. Bluetooth™ and other wireless media. These communication media can be used for communications units 144, 154 or as antenna 112 or as an alternative or supplement to antenna 112.

Turning to the control module 102, some of the components (for example, a power source 114, an antenna 112, and a processor 110) of the electrical stimulation system can be positioned on one or more circuit boards or similar carriers within a sealed housing of the control module (implantable pulse generator,) if desired. Any power source 114 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the antenna 112 or a secondary antenna. The external power source can be in a device that is mounted on the skin of the user or in a unit that is provided near the user on a permanent or periodic basis.

If the power source 114 is a rechargeable battery, the battery may be recharged using the antenna 112, if desired. Power can be provided to the battery for recharging by inductively coupling the battery through the antenna to a recharging unit external to the user.

A stimulation signal, such as electrical current in the form of electrical pulses, is emitted by the electrodes of the lead 108 (or a microstimulator) to stimulate neurons, nerve fibers, muscle fibers, or other body tissues near the electrical stimulation system. Examples of leads are described in more detail below. The processor 110 is generally included to control the timing and electrical characteristics of the electrical stimulation system. For example, the processor 110 can, if desired, control one or more of the timing, frequency, strength, duration, and waveform of the pulses. In addition, the processor 110 can select which electrodes can be used to provide stimulation, if desired. In some embodiments, the processor 110 selects which electrode(s) are cathodes and which electrode(s) are anodes. In some embodiments, the processor 110 is used to identify which electrodes provide the most useful stimulation of the desired tissue.

With respect to the control module 102, external programming unit 106, and remote data storage unit 104, any suitable processor 110, 140, 150 can be used in these devices. For the control module 102, the processor 110 is capable of receiving and interpreting instructions from an external programming unit 106a, 106b that, for example, allows modification of pulse characteristics. In the illustrated embodiment, the processor 110 is coupled to the antenna 112. This allows the processor 110 to receive instructions from the external programming unit 106a, 106b to, for example, direct the pulse characteristics and the selection of electrodes, if desired. The antenna 112, or any other antenna described herein, can have any suitable configuration including, but not limited to, a coil, looped, or loopless configuration, or the like.

In one embodiment, the antenna 112 is capable of receiving signals (e.g., RF signals) from the external programming unit 106a, 106b. The external programming unit 106a. 106b can be a home station or unit at a clinician's office or any other suitable device. In some embodiments, the external programming unit 106a, 106b can be a device that is worn on the skin of the user or can be carried by the user and can have a form similar to a pager, cellular phone, or remote control, if desired. The external programming unit 106a, 106b can be any unit that can provide information to the control module 102. One example of a suitable external programming unit 106a, 106b is a computer operated by the user or clinician to send signals to the control module 102. Another example is a mobile device or an application on a mobile device that can send signals to the control module 102

The signals sent to the processor 110 via the antenna 112 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the control module 102 to cease operation, to start operation, to start charging the battery, or to stop charging the battery.

Optionally, the control module 102 may include a transmitter (not shown) coupled to the processor 110 and the antenna 112 for transmitting signals back to the external programming unit 106a, 106b or remote data storage 104 or another unit capable of receiving the signals. For example, the control module 102 may transmit signals indicating whether the control module 102 is operating properly or not or indicating when the battery needs to be charged or the level of charge remaining in the battery. The processor 110 may also be capable of transmitting information about the pulse characteristics so that a user or clinician can determine or verify the characteristics.

Any suitable memory 116, 142, 152 can be used for the respective components of the system 100. The memory 116, 142, 152 illustrates a type of computer-readable media, namely computer-readable storage media. Computer-readable storage media may include, but is not limited to, nonvolatile, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal.” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The user interface 156 of the external programming unit 106 and optional user interface 146 of the remote data storage unit 104 can be, for example, a keyboard, mouse, touch screen, track ball, joystick, voice recognition system, or any combination thereof, and the like.

There are a variety of methods for synchronizing stimulation data between external programming units, control modules, and remote data storage units. The stimulation data that is synchronized can include, but is not limited to, stimulation parameters that are used by the control module to create the stimulation signal provided to patient tissue through the electrodes, stimulation results measured by sensors or entered by a clinician or user or obtained in any other manner, patient information, status of the control module, battery status, and the like. The stimulation data may also include a history of changes, addition, and deletions to the stimulation data.

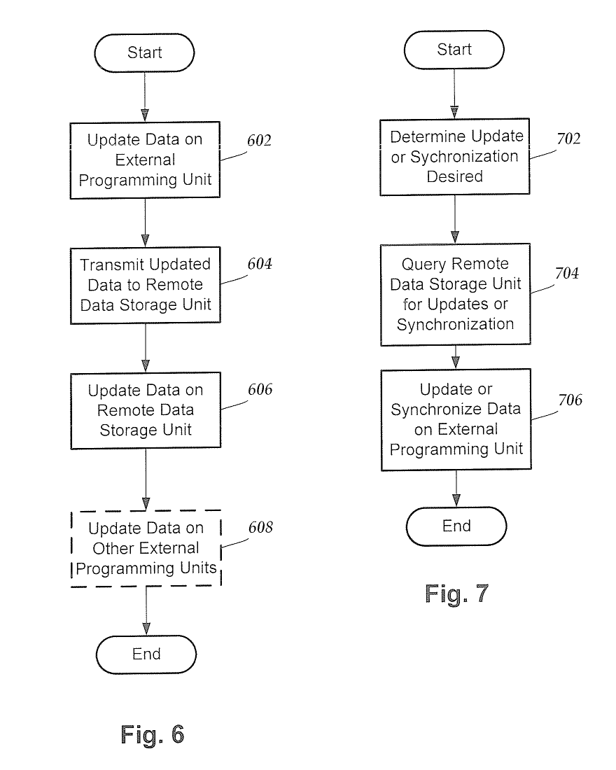

FIG. 6 is a flowchart of one method for synchronizing stimulation data between an external programming unit (such as external programming units 106a. 106b of FIGS. 1-3) and a remote data storage unit (such as remote data storage unit 104 of FIGS. 1 and 2 and external programming unit 106a of FIG. 3). In step 602, the stimulation data is updated or otherwise changed on the external programming unit. The updating of the stimulation data can include, for example, entering initial stimulation parameters, changing (including adding or deleting) stimulation parameters, receiving sensor data from the control module or another sensor, receiving clinician or patient feedback about the stimulation, receiving or changing patient data, or the like.

In step 604, the external programming unit transmits the updated data to the remote data storage unit using any suitable communication method or combination of communication methods, as described above. In step 606, the remote data storage unit stores the updated data. In some embodiments, the remote data storage unit may maintain a history of changes to the stimulation data for a particular control module or patient or both.

Optionally, the remote data storage unit can push (e.g., transmit or otherwise deliver) the updated stimulation data to other external programming units in step 608. Alternatively or additionally, other external programming units can query the remote data storage unit for any updated stimulation data.

FIG. 7 is a flowchart of one method of requesting an update or synchronization from a remote data storage unit (such as remote data storage unit 104 of FIGS. 1 and 2 and external programming unit 106a of FIG. 3) by the external programming unit (such as external programming units 106a, 106b of FIGS. 1-3). In step 702, it is determined that an update or synchronization of stimulation data is desired. This determination may occur when the external programming unit is turned on. Alternatively or additionally, a determination may be made upon user request, for example, at the beginning, or during, an implantation procedure or a control unit programming procedure. In some embodiments, the external programming unit may additionally or alternatively request updating or synchronization on a periodic basis, for example, every hour, every day, every week, every month, or any other appropriate time interval.

In step 704, the external programming unit queries the remote data storage unit for updated stimulation data or synchronization. In some embodiments, the query may include the time or date of the last update or synchronization. In at least some embodiments, the query may specify a portion, but less than all, of the stimulation data that is to be updated or synchronized.

In step 706, in response to the query the external programming unit receives the updated or synchronized stimulation data from the remote data storage unit and stores that data. In some embodiments, the external programming unit only receives stimulation data that has been updated since the date of the last update or synchronization provided. This date is optionally provided by the external programming unit. In some embodiments, the external programming unit receives all of the requested stimulation data and replaces the previous stimulation data with the received stimulation data. In some embodiments, the external programming unit receives all of the requested stimulation data, but only replaces portions of the previous stimulation data, for example, only those portions that have been updated or otherwise changed, added, or subtracted.

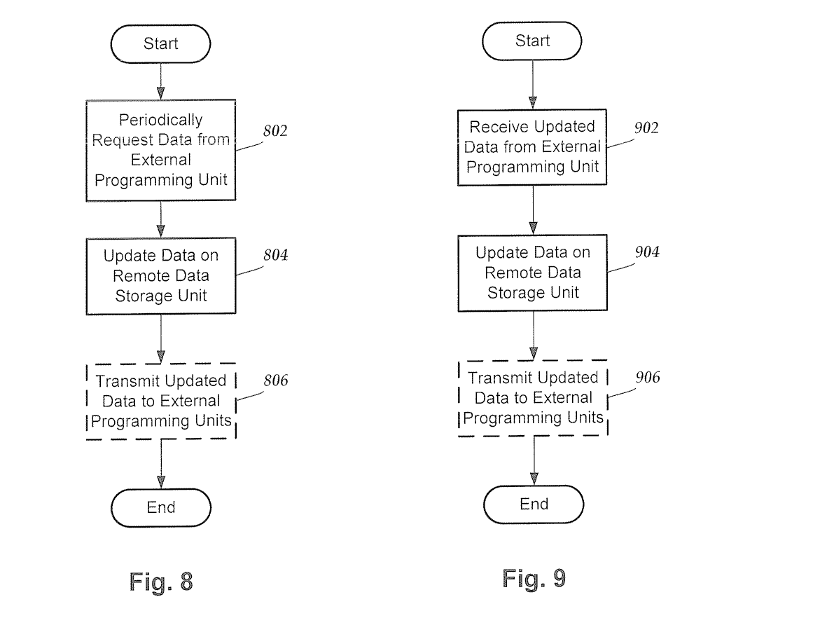

FIG. 8 is a flowchart of one method for a remote data storage unit (such as remote data storage unit 104 of FIGS. 1 and 2 and external programming unit 106a of FIG. 3) to update or synchronize stimulation data with one or more external programming units (such as external programming units 106a. 106b of FIGS. 1-3). In step 802, the remote data storage unit (or an external programming unit that stores the stimulation data, see FIG. 3) periodically requests stimulation data from one or more external programming units. The period between requests can be any suitable period including, but not limited to, one hour, six hours, twelve hours, one day, two days, one week, two weeks, one month, three months, six months, one year, or the like. In at least some embodiments, a clinician or other user can set the period. In some embodiments, one or more external programming units are associated with the remote data storage unit and the remote data storage unit requests the stimulation data from the external programming unit(s). In some embodiments, one or more external programming units are associated with a particular patient or a particular control module and the remote data storage unit requests the stimulation data related to that patient or control module from the external programming unit(s). In some embodiments, if a particular external programming unit does not respond to the request, the remote data storage unit can schedule a follow-up request for the stimulation data from that external programming unit after a follow-up period (for example, one hour, two hours, one day, two days, one week, one month, or any other suitable period.) In some embodiments, if a particular external programming unit does not respond to the request, the remote data storage unit can invoke an alarm or send a warning message to the clinician or any other suitable entity indicating the failure to receive a response from the external programming unit.

In step 804, updated stimulation data is sent from the external programming unit(s) to the remote data storage unit and the stimulation data on the remote data storage unit is updated. In some embodiments, each external programming unit may send all of the stimulation data to the remote data storage unit. In other embodiments, the external data programming unit may only send stimulation data that has been added or changed since the last query by the remote data storage unit or since a date specified by the remote data storage unit in its request. In some embodiments, the remote data storage unit may analyze the stimulation data or portions of the stimulation data to determine whether it is more recent than that currently stored at the remote data storage unit or that received from another external programming unit prior to updating the stimulation data on the remote data storage unit.

In optional step 806, the remote data storage unit may push (e.g., transmit or otherwise deliver) the updated stimulation data to one or more (or even all) of the external programming units.

FIG. 9 is a flowchart of another method for a remote data storage unit (such as remote data storage unit 104 of FIGS. 1 and 2 and external programming unit 106a of FIG. 3) to update or synchronize stimulation data with one or more external programming units (such as external programming units 106a, 106b of FIGS. 1-3). In step 902, the remote data storage unit (or an external programming unit that stores the stimulation data, see FIG. 3) determines whether an update or synchronization of the stimulation data is desired. This determination can be the result of a triggering event such as, but not limited to, a system interrupt, a notification of the availability of new or updated stimulation data, a user command to update or synchronize stimulation data, addition of a new control module or external programming unit to the system, or the like. In some embodiments, one or more external programming units are associated with the remote data storage unit and the remote data storage unit requests the stimulation data from the external programming unit(s). In some embodiments, one or more external programming units are associated with a particular patient or a particular control module and the remote data storage unit requests the stimulation data related to that patient or control module from the external programming unit(s). In some embodiments, if a particular external programming unit does not respond to the request, the remote data storage unit can schedule a follow-up request for the stimulation data from that external programming unit after a follow-up period (for example, one hour, two hours, one day, two days, one week, one month, or any other suitable period.) In some embodiments, if a particular external programming unit does not respond to the request, the remote data storage unit can invoke an alarm or send a warning message to the clinician or any other suitable entity indicating the failure to receive a response from the external programming unit.

In step 904, updated stimulation data is sent from the external programming unit(s) to the remote data storage unit and the stimulation data on the remote data storage unit is updated. In some embodiments, each external programming unit may send all of the stimulation data to the remote data storage unit. In other embodiments, the external data programming unit may only send stimulation data that has been added or changed since the last query by the remote data storage unit or since a date specified by the remote data storage unit in its request. In some embodiments, the remote data storage unit may analyze the stimulation data or portions of the stimulation data to determine whether it is more recent than that currently stored at the remote data storage unit or that received from another external programming unit prior to updating the stimulation data on the remote data storage unit.

In optional step 906, the remote data storage unit may push the updated stimulation data to one or more (or even all) of the external programming units.

It will be understood that the system can include one or more of the methods described hereinabove with respect to FIGS. 6-9 in any combination. The methods, systems, and units described herein may be embodied in many different forms and should not be construed as limited to the embodiments set forth herein. Accordingly, the methods, systems, and units described herein may take the form of an entirely hardware embodiment, an entirely software embodiment or an embodiment combining software and hardware aspects. The methods described herein can be performed using any type of processor or any combination of processors where each processor performs at least part of the process.

It will be understood that each block of the flowchart illustrations, and combinations of blocks in the flowchart illustrations and methods disclosed herein, can be implemented by computer program instructions. These program instructions may be provided to a processor to produce a machine, such that the instructions, which execute on the processor, create means for implementing the actions specified in the flowchart block or blocks or described for the control modules, external programming units, remote data storage units, systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

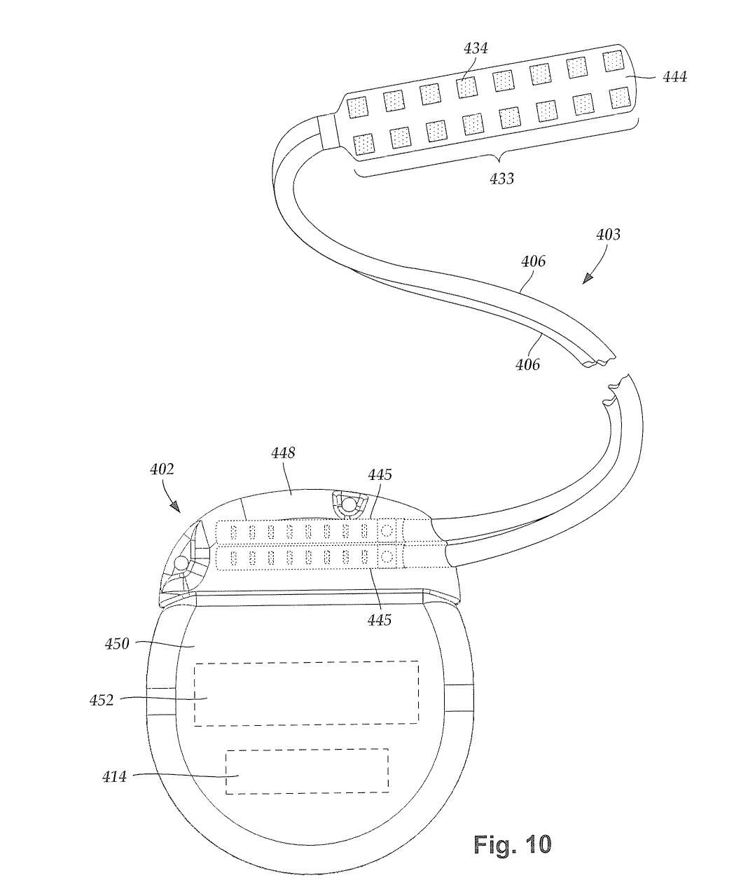

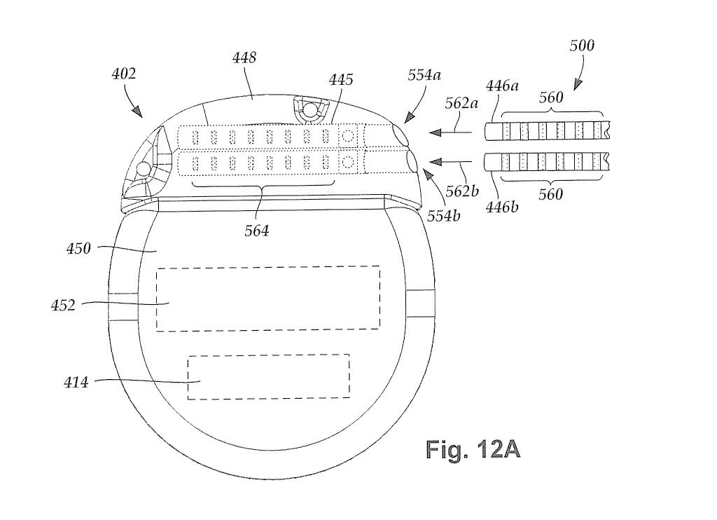

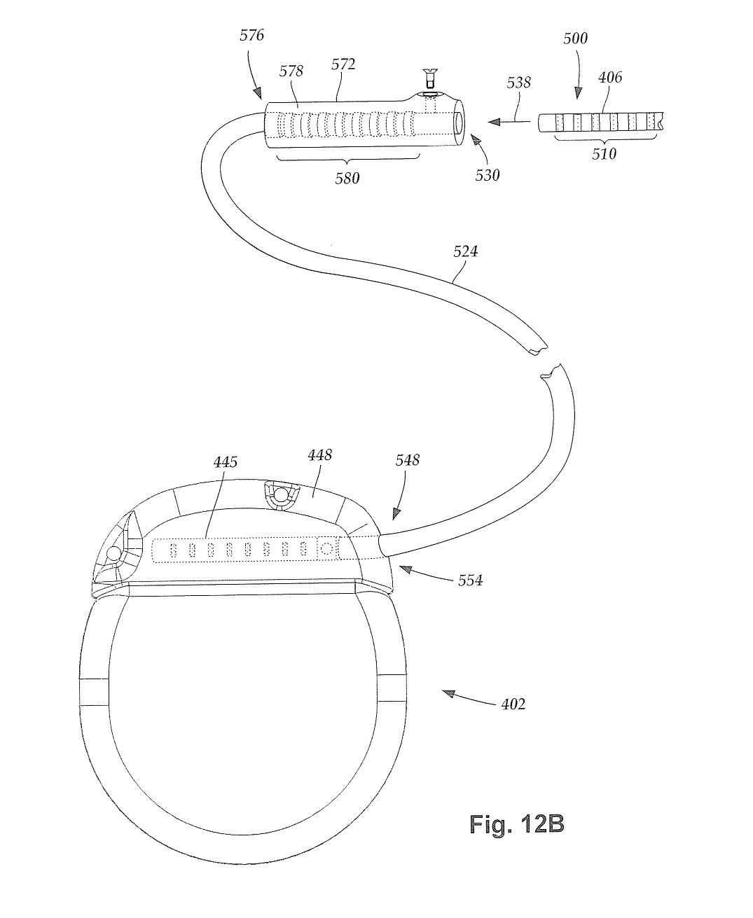

FIG. 10 illustrates one embodiment of a control module 402 and lead 403. The lead 403 includes a paddle body 444 and one or more lead bodies 446. In FIG. 10, the lead 403 is shown having two lead bodies 446. It will be understood that the lead 403 can include any suitable number of lead bodies including, for example, one, two, three, four, five, six, seven, eight or more lead bodies 446. An array of electrodes 433, such as electrode 434, is disposed on the paddle body 444, and one or more terminals (e.g., 560 in FIGS. 12A and 12B) are disposed along each of the one or more lead bodies 446. In at least some embodiments, the lead has more electrodes than terminals.

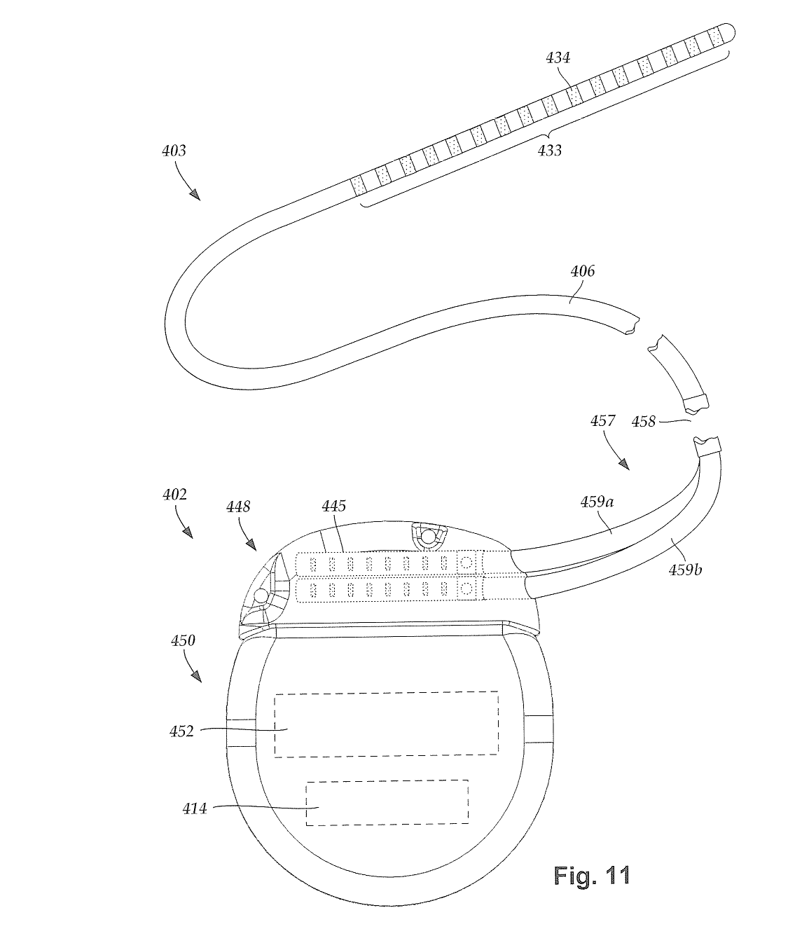

FIG. 11 illustrates schematically another embodiment in which the lead 403 is a percutaneous lead. In FIG. 11, the electrodes 434 are shown disposed along the one or more lead bodies 446. In at least some embodiments, the lead 403 is isodiametric along a longitudinal length of the lead body 446.

The lead 403 can be coupled to the implantable control module 402 in any suitable manner. In FIG. 10, the lead 403 is shown coupling directly to the implantable control module 402. In at least some other embodiments, the lead 403 couples to the implantable control module 402 via one or more intermediate devices (500 in FIGS. 12A and 12B). For example, in at least some embodiments one or more lead extensions 524 (see e.g., FIG. 12B) can be disposed between the lead 403 and the implantable control module 402 to extend the distance between the lead 403 and the implantable control module 402. Other intermediate devices may be used in addition to, or in lieu of, one or more lead extensions including, for example, a splitter, an adaptor, or the like or combinations thereof. It will be understood that, in the case where the electrical stimulation system includes multiple elongated devices disposed between the lead 403 and the implantable control module 402, the intermediate devices may be configured into any suitable arrangement.

In FIG. 11, the electrical stimulation system 400 is shown having a splitter 457 configured and arranged for facilitating coupling of the lead 403 to the implantable control module 402. The splitter 457 includes a splitter connector 458 configured to couple to a proximal end of the lead 403, and one or more splitter tails 459a and 459b configured and arranged to couple to the implantable control module 402 (or another splitter, a lead extension, an adaptor, or the like).

The implantable control module 402 includes a connector housing 448 and a sealed electronics housing 450. An electronic subassembly 452 (which can include the processor 110 (see. FIGS. 1-3)) and the power source 414 are disposed in the electronics housing 450. A connector 445 is disposed in the connector housing 448. The connector 445 is configured and arranged to make an electrical connection between the lead 403 and the electronic subassembly 452 of the implantable control module 402.

The electrical stimulation system or components of the electrical stimulation system, including the paddle body 444, the one or more of the lead bodies 446, and the implantable control module 402, are typically implanted into the body of a patient. The electrical stimulation system can be used for a variety of applications including, but not limited to deep brain stimulation, neural stimulation, spinal cord stimulation, muscle stimulation, and the like.

The electrodes 434 can be formed using any conductive, biocompatible material. Examples of suitable materials include metals, alloys, conductive polymers, conductive carbon, and the like, as well as combinations thereof. In at least some embodiments, one or more of the electrodes 434 are formed from one or more of: platinum, platinum iridium, palladium, palladium rhodium, or titanium.

Any suitable number of electrodes 434 can be disposed on the lead including, for example, four, five, six, seven, eight, nine, ten, eleven, twelve, fourteen, sixteen, twenty-four, thirty-two, or more electrodes 434. In the case of paddle leads, the electrodes 434 can be disposed on the paddle body 444 in any suitable arrangement. In FIG. 10, the electrodes 434 are arranged into two columns, where each column has eight electrodes 434.

The electrodes of the paddle body 444 (or one or more lead bodies 446) are typically disposed in, or separated by, a non-conductive, biocompatible material such as, for example, silicone, polyurethane, polyetheretherketone (“PEEK”), epoxy, and the like or combinations thereof. The one or more lead bodies 446 and, if applicable, the paddle body 444 may be formed in the desired shape by any process including, for example, molding (including injection molding), casting, and the like. The non-conductive material typically extends from the distal ends of the one or more lead bodies 446 to the proximal end of each of the one or more lead bodies 446.

In the case of paddle leads, the non-conductive material typically extends from the paddle body 444 to the proximal end of each of the one or more lead bodies 446. Additionally, the non-conductive, biocompatible material of the paddle body 444 and the one or more lead bodies 446 may be the same or different. Moreover, the paddle body 444 and the one or more lead bodies 446 may be a unitary structure or can be formed as two separate structures that are permanently or detachably coupled together.

One or more terminals (e.g., 560 in FIGS. 12A-12B) are typically disposed along the proximal end of the one or more lead bodies 446 of the electrical stimulation system 400 (as well as any splitters, lead extensions, adaptors, or the like) for electrical connection to corresponding connector contacts (e.g., 564 in FIGS. 12A-12B). The connector contacts are disposed in connectors (e.g., 445 in FIGS. 4-12B; and 572 FIG. 12B) which, in turn, are disposed on, for example, the implantable control module 402 (or a lead extension, a splitter, an adaptor, or the like). One or more electrically conductive wires, cables, or the like (i.e., “conductors”—not shown) extend from the terminal(s) to the electrode(s). In at least some embodiments, there is at least one (or exactly one) terminal conductor for each terminal which extends to at least one (or exactly one) of the electrodes.

The one or more conductors are embedded in the non-conductive material of the lead body 446 or can be disposed in one or more lumens (not shown) extending along the lead body 446. For example, any of the conductors may extend distally along the lead body 446 from the terminals 560.

FIG. 12A is a schematic side view of one embodiment of a proximal end of one or more elongated devices 500 configured and arranged for coupling to one embodiment of the connector 445. The one or more elongated devices may include, for example, one or more of the lead bodies 446 of FIG. 10, one or more intermediate devices (e.g., a splitter, the lead extension 524 of FIG. 12B, an adaptor, or the like or combinations thereof), or a combination thereof.

The connector 445 defines at least one port into which a proximal ends 446A, 446B of the elongated device 500 can be inserted, as shown by directional arrows 562a. 562b. In FIG. 12A (and in other figures), the connector housing 448 is shown having two ports 554a, 554b. The connector housing 448 can define any suitable number of ports including, for example, one, two, three, four, five, six, seven, eight, or more ports.

The connector 445 also includes one or more connector contacts, such as connector contact 564, disposed within each port 554a, 554b. When the elongated device 500 is inserted into the ports 554a, 554b, the connector contact(s) 564 can be aligned with the terminal(s) 560 disposed along the proximal end(s) of the elongated device(s) 500 to electrically couple the implantable control module 402 to the electrodes (434 of FIG. 10) disposed on the paddle body 445 of the lead 403. Examples of connectors in implantable control modules are found in, for example, U.S. Pat. Nos. 7,244,150 and 8,224,450, which are incorporated by reference.

FIG. 12B is a schematic side view of another embodiment that includes a lead extension 524 that is configured and arranged to couple one or more elongated devices 500 (e.g., one of the lead bodies 446 of FIGS. 10 and 11, the splitter 457 of FIG. 11, an adaptor, another lead extension, or the like or combinations thereof) to the implantable control module 402. In FIG. 12B, the lead extension 524 is shown coupled to a single port 554 defined in the connector 445. Additionally, the lead extension 524 is shown configured and arranged to couple to a single elongated device 500. In alternate embodiments, the lead extension 524 is configured and arranged to couple to multiple ports 554 defined in the connector 445, or to receive multiple elongated devices 500, or both.

A lead extension connector 572 is disposed on the lead extension 524. In FIG. 12B, the lead extension connector 572 is shown disposed at a distal end 576 of the lead extension 524. The lead extension connector 572 includes a connector housing 578. The connector housing 578 defines at least one port 530 into which terminal(s) 560 of the elongated device 500 can be inserted, as shown by directional arrow 538. The connector housing 578 also includes a plurality of connector contacts, such as connector contact 580. When the elongated device 500 is inserted into the port 530, the connector contacts 580 disposed in the connector housing 578 can be aligned with the terminal(s) 560 of the elongated device 500 to electrically couple the lead extension 524 to the electrodes (434 of FIGS. 10 and 11) disposed along the lead (403 in FIGS. 10 and 11).

In at least some embodiments, the proximal end of the lead extension 524 is similarly configured and arranged as a proximal end of the lead 403 (or other elongated device 500). The lead extension 524 may include one or more electrically conductive wires (not shown) that electrically couple the connector contact(s) 580 to a proximal end 548 of the lead extension 524 that is opposite to the distal end 576. The conductive wire(s) disposed in the lead extension 524 can be electrically coupled to one or more terminals (not shown) disposed along the proximal end 548 of the lead extension 524. The proximal end 548 of the lead extension 524 is configured and arranged for insertion into a connector disposed in another lead extension (or another intermediate device). As shown in FIG. 12B, the proximal end 548 of the lead extension 524 is configured and arranged for insertion into the connector 445.

The embodiments of FIGS. 10-12B illustrate a control module 402 with a connector 445 into which a proximal end portion of the lead or lead extension can be removably inserted. It will be recognized, however, that other embodiments of a control module and lead can have the lead or lead extension permanently attached to the control module. Such an arrangement can reduce the size of the control module as the conductors in the lead can be permanently attached to the electronic subassembly. It will also be recognized that, in at least some embodiments, more than one lead can be attached to a control module.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation system, comprising:
an implantable control module configured and arranged for implantation in a body of a patient and comprising an antenna and a processor coupled to the antenna, wherein the control module is configured and arranged to provide electrical stimulation signals to an electrical stimulation lead coupled to the implantable control module for stimulation of patient tissue; and
a first external programming unit configured and arranged to communicate with the processor of the implantable control module using the antenna and to provide or update stimulation parameters for production of the electrical stimulation signals, wherein the first external programming unit is configured and arranged to communicate with a data storage unit remote from the first external programming unit and the implantable control module to store the stimulation parameters at the data storage unit and retrieve the stimulation parameters from the data storage unit.

2. The electrical stimulation system of claim 1, further comprising the data storage unit remote from the first external programming unit and the implantable control module and configured and arranged to communicate with the external programming unit to store the stimulation parameters and to allow retrieval of the stimulation parameters.

3. The electrical stimulation system of claim 2, wherein the control module and data storage unit are configured and arranged to communicate directly with each other.

4. The electrical stimulation system of claim 2, further comprising a second external programming unit configured and arranged to communicate with the control module and the data storage unit and to provide or update stimulation parameters for production of the electrical stimulation signals.

5. The electrical stimulation system of claim 2, wherein the data storage unit is a second external programming unit that is configured and arranged to communicate with the processor of the implantable control module using the antenna and to provide or update stimulation parameters for production of the electrical stimulation signals.

6. The electrical stimulation system of claim 1, wherein the external programming unit, comprises
a user interface configured and arranged to receive input from a user, and
a processor in communication with the user interface and configured and arranged to perform the following actions:
a) receiving input from the user to change or add at least one stimulation parameter,
b) in response to receiving the input, communicating the at least one stimulation parameter to the implantable control module, and
c) in response to receiving the input, communicating the at least one stimulation parameter to the data storage unit for storing or updating the at least one stimulation parameter.

7. The electrical stimulation system of claim 6, wherein the processor is configured and arranged to perform the following additional action:
d) retrieving a plurality of stimulation parameters from the data storage unit in preparation for receiving input from the user regarding at least one stimulation parameter.

8. The electrical stimulation system of claim 2, wherein the data storage unit, comprises
a memory, and
a processor in communication with the memory and configured and arranged to perform the following actions:
a) requesting stimulation data from at least one external programming unit, wherein the stimulation data comprises at least one stimulation parameter,
b) in response to requesting the stimulation data, receiving the stimulation data from the at least one external programming unit, and
c) in response to receiving the stimulation data, using the stimulation data to update stimulation data stored in the memory.

9. The electrical stimulation system of claim 8, wherein the action of requesting stimulation data comprises requesting the stimulation data from at least one external programming unit on a regular periodic basis.

10. The electrical stimulation system of claim 8, wherein the action of requesting stimulation data comprises requesting the stimulation data from at least one external programming unit in response to a triggering event.

11. The electrical stimulation system of claim 8, wherein the processor is configured and arranged to perform the following additional action:
d) in response to receiving the stimulation data, communicating at least a portion of the stimulation data to at least one of the at least one external programming unit.

12. A non-transitory computer-readable medium having processor-executable instructions for providing or updating stimulation parameters of an electrical stimulation system, the processor-executable instructions when installed onto a device enable the device to perform actions, comprising:
receiving input from the user to change or add at least one stimulation parameter;
in response to receiving the input, communicating the at least one stimulation parameter to an implantable control module; and
in response to receiving the input, communicating the at least one stimulation parameter to a data storage unit for storing or updating the at least one stimulation parameter, wherein the data storage unit is remote from the control module and from the device performing the actions.

13. The non-transitory computer-readable medium of claim 12, wherein the processor-executable instructions when installed onto a device enable the device to perform the following additional action:
retrieving a plurality of stimulation parameters from the data storage unit in preparation for receiving input from the user regarding at least one stimulation parameter.

14. The non-transitory computer-readable medium of claim 12, wherein retrieving a plurality of stimulation parameters comprises requesting an update of the plurality of stimulation parameters with a date of a last update and retrieving those stimulation parameters that have changed since the date of the last update.

15. A non-transitory computer-readable medium having processor-executable instructions for providing or updating stimulation parameters of an electrical stimulation system, the processor-executable instructions when installed onto a device enable the device to perform actions, comprising:
requesting stimulation data from at least one external programming unit, wherein the stimulation data comprises at least one stimulation parameter;
in response to requesting the stimulation data, receiving the stimulation data from the at least one external programming unit; and
in response to receiving the stimulation data, using the stimulation data to update stimulation data stored in the memory.

16. The non-transitory computer-readable medium of claim 15, wherein the action of requesting stimulation data comprises requesting the stimulation data from at least one external programming unit on a regular periodic basis.

17. The non-transitory computer-readable medium of claim 15, wherein the action of requesting stimulation data comprises requesting the stimulation data from at least one external programming unit in response to a triggering event.

18. The non-transitory computer-readable medium of claim 15, wherein the processor-executable instructions when installed onto a device enable the device to perform the following additional action:
in response to receiving the stimulation data, communicating at least a portion of the stimulation data to at least one of the at least one external programming unit.

19. The non-transitory computer-readable medium of claim 15, wherein the processor-executable instructions when installed onto a device enable the device to perform the following additional action:
maintaining a history of changes to the stimulation data.

20. The non-transitory computer-readable medium of claim 15, wherein the processor-executable instructions when installed onto a device enable the device to perform the following additional action:
in response to requesting the stimulation data, if an external programming unit does not respond, sending a follow-up request for the stimulation data.
